(19) United States
(12) Patent Application Publication
(10) Pub. No.: US 2013/0261374 A1
(43) Pub. Date: Oct. 3, 2013

(54) DEVICE AND METHOD FOR GENERATING MAGNETIC FIELDS

(71) Applicant: Iain Elder, Suffolk (GB)

(72) Inventor: Iain Elder, Suffolk (GB)

(73) Assignee: Pulse Medical Technologies Ltd. Institute of Life Science Swansea University, Swansea (GB)

(21) Appl. No.: 13/990,559

(22) PCT Filed: Nov. 30, 2011
(86) PCT No.: PCT/GB2011/001663

§ 371 Date: May 30, 2013

(30) Foreign Application Priority Data
Nov. 30, 2010 (GB) 1020266.1

(51) Int. Cl. A61N 2/00

(52) U.S. Cl. CPC A61N 2/00
USPC 600/15; 600/9

(57) ABSTRACT

This invention relates to an improved magnetic field induction therapy device and an improved method of treatment using magnetic field induction therapy. The method and device utilise pulsed alternating magnetic fields of specific characteristics which have been found to be, at least, therapeutic, In particular, the present invention relates to a device and method for creating varied and programmable magnetic fields, especially in a hand held unit for medical or therapeutic applications. The therapeutic value of magnetic fields has been known for more than two thousand years. Permanent magnets placed on or adjacent an animal or human body have been used for pain relief and blood circulation improvement. It is also known to use pulsating magnetic fields for medical and therapeutic purposes. For over 40 years the use of electro-magnetic fields has been known to be more beneficial than static or permanent magnetic fields. Pulsed and variable electro-magnetic Field Therapy (PEMFT) provide an alternative to conventional methods for treatment of certain medical conditions. PEMFT is particularly, but not exclusively, used to treat pain and wound healing. Typically, all or part of a patient's body is exposed to a pulsating electro-magnetic field, the field being characterised by its variable pulse frequency and its magnetic flux density (usually measured in Tesla), or equivalent field strength measure. It is believed that the characteristics of the magnetic field have an effect on the effectiveness of the treatment. It is an object of the present invention to provide an apparatus and method for creating pulsed and variable electromagnetic fields which is convenient to use and relatively effective in its treatment.

### FIELD OF THE INVENTION

This invention relates to an improved magnetic field induction therapy device and an improved method of treatment using magnetic field induction therapy. The method and device utilise pulsed alternating magnetic fields of specific characteristics which have been found to be, at least, therapeutic. In particular, the present invention relates to a device and method for creating varied and programmable magnetic fields, especially in a hand held unit for medical or therapeutic applications.

### BACKGROUND TO THE INVENTION

The therapeutic value of magnetic fields has been known for more than two thousand years. Permanent magnets placed on or adjacent an animal or human body have been used for pain relief and blood circulation improvement. It is also known to use pulsating magnetic fields for medical and therapeutic purposes. For over 40 years the use of electro-magnetic fields has been known to be more beneficial than static or permanent magnetic fields. The most common devices use a generator unit to produce a pulsing or time-varying electric current which is fed into a wire-wound coil or coils to produce an electromagnetic field.

In most commercially available prior art devices direct current (D.C.) pulses are used to energise the applicator coils. These prior art devices have proven to be effective in providing temporary pain relief and promoting repair of damaged tissue. The inventors have found that an enhanced effect can be obtained by applying multi-rhythm bio-waveforms of pulsed alternating magnetic fields. Pulsed and variable electro-magnetic Field Therapy (PEMFT) provide an alternative to conventional methods for treatment of certain medical conditions. PEMFT is particularly, but not exclusively, used to treat pain and wound healing. Typically, all or part of a patient's body is exposed to a pulsating electro-magnetic field, the field being characterised by its variable pulse frequency and its magnetic flux density (usually measured in Tesla), or equivalent field strength measure. It is believed that the characteristics of the magnetic field have an effect on the effectiveness of the treatment. As is known, a time-varying electric field generates a time-varying magnetic field and vice versa. Therefore, as an oscillating electric field generates an oscillating magnetic field, the magnetic field in turn generates an oscillating electric field, and so on.

Conventional devices for delivering Pulsed Electro-Magnetic Fields (PEMFs) tend to be large and cumbersome and do not readily lend themselves to private personal use. U.S. Pat. No. 3,658,051 (Maclean et al) teaches of a method of treatment including positioning a part of a patient to be treated between the poles of an electromagnet. The patient part is then subjected to a pulsating magnetic field induced in the electromagnet by an intermittent direct current, the peak intensity of each pulse being at least 0.2 Tesla. Preferably each pulse has a duration of at least 0.25 seconds, and about one pulse per 0.5 seconds is administered. This arrangement is, however, rather large; a cabinet is employed together with pole pieces which are arranged to surround a patient body part such as a limb etc.

U.S. Pat. No. 5,387,176 (Markoll et al) provides an arrangement and process for treating acute diseases of a body organ of the musculoskeletal system by applying a magnetic field by means of an annular coil surrounding the diseased organ, the coil being energized by a pure DC voltage having a rectangular wave form pulsing at the rate of 1-30 cps. The field at the target organ is of low intensity, preferably under 20 gauss, and the field lines are oriented, where the target organ is on an appendage, such that the flux lines travel toward the distal end of the appendage. This system comprises a large wound circular toroid similar to a large bangle for wrist and arm treatments, having a diameter of 16 cm or so; with larger diameter wound toroids for lower limb application and whole body application, where the diameters are typically around 27 cm and 55 cm in diameter.

U.S. Pat. No. 7,175,587 provides a hand-held—yet corded—mains powered device. A power and timer circuit supplies current pulses that approximate square pulses in form, so that the straight wire element generates magnetic pulses having rapid rise and fall times. Peak field strength is approximately 2 gauss at a 1 cm distance from the magnetic field inducing element. Another known system employs a magnetic coil of a pad type having an outside diameter of 170 mm and approximately 800 turns of copper wire with an impedance of preferably 15 Ohms. Not only is this large and relatively non-specific in application, current drain is significant and a mobile device has not been possible.

The use of PEMF can provide a very efficient and simple therapy method. Indications vary from muscular cramps—both spinal and relating to limbs, the therapy with pulsating magnetic fields (PEMF) provides a biophysical modality used in medicine for accelerated therapeutic purposes. It is believed that the body is influenced either generally or locally with magnetic field impulses. Body cellular functions can be improved considerably. The pulsating magnetic field has a high biological effectiveness, which is being used in the medical field as a means of therapy as well as in the area of diagnostics.

Human and animal bodies are insignificantly diamagnetic and paramagnetic i.e. they are neutral to a reduction (divergence of magnetic field lines) or a concentration (convergence of Magnetic field lines) of magnetic field, as is air. The consequence of this is that whenever a magnetic field passes through an animal organism or on parts of it, the magnetic field absolutely permeates such organism or parts thereof, i.e. within a standard range of magnetic fields, all parts of the body are penetrated completely by the field lines. Whilst there is no magnetic field absorption medium as such, one can be shielded against the effects of a magnetic field by the use strong ferromagnetic materials, where the field is said to be shunted shunting them into strong ferromagnetic materials. Therefore, it is difficult to produce field-free spaces when magnetic fields pass through a material.

It is known that the human and animal organism consists of a large number of cells which function, in part, due to differences in electrical potential.

These cells are functioning electrically. If there is no electrical potential left in the cell, it is no longer viable. These cells have a basic (or rest) potential that is necessary for normal cellular metabolism. Diseased or damaged cells can have an altered rest potential. These cells have ions—electrically charged particles surrounding the cells—which, when a pulsating magnetic fields is applied, will be influenced by such pulsating magnetic fields. The rest potential of a cell is proportional to an extent to ion exchange occurring at the cell membrane. The ion exchange is also, in part, responsible for the oxygen utilization of the cell. It is known that pulsating magnetic fields can dramatically influence the ion exchange at the cellular level and thereby, it is believed, greatly improve the oxygen utilization of diseased or damaged tissues. The deterioration of the oxygen utilization is known to be a problem in several medical branches, especially delayed healing and arthritis of joints. The basis of magnetic therapy is that artificially produced magnetic fields can have a beneficial effect on living tissue by interaction with the paramagnetic components.

Electrical potential differences exist across cell walls. Such potential differences are maintained by the movement of ions and in particular, sodium, potassium, chromium and HCO−. Normal cell potentials vary from −60 mV in some muscles to as much as −120 mV in myocardial cells. This movement of ions is known to have a profound influence on the inflammatory process of living tissue. Cell wall potential differences tend to attract positive ions into and negative ions out of the cell, resulting in a reduction in cell potential, an increase in fluid (oedema) and a triggering, under certain conditions, of pain mechanisms. A pulsed alternating magnetic field can influence the net ion flux through the cell membrane to restore the normal cell potential and Na+ and K+ balance, and release encephalin, endorphin and other chemicals to inhibit or modify pain signal transmission.

### OBJECT TO THE INVENTION

The present invention seeks to provide an improved method and apparatus which provide therapeutic effects by the use of pulsed alternating magnetic fields applied to living tissue.

The present invention also seeks to provide an apparatus and method for creating pulsed and variable electro-magnetic fields which is convenient to use and relatively effective in its treatment.

### STATEMENT OF INVENTION

Generally, in a first aspect of the invention provides a device for generating a variable pulsed and variable electro-magnetic field, the device comprising a plurality of reactive loads, e.g. inductive loads, each load being located between first and second load terminals; and means for causing at least part of a pulsating DC voltage to be applied across said first and second terminals in a pulsed manner.

In the preferred embodiment, the device is arranged to generate a pulsed and varying electro-magnetic field having a peak amplitude in the region of 1 G-100 G, preferably between 10 G and 50 G, advantageously at a frequency of between 1 kHz and 25 kHz, preferably approximately 20 kHz. The pulsed and varying electro-magnetic field advantageously includes a frequency deviation contained within a carrier signal, the carrier preferably having a frequency of approximately 20 kHz. The unit may also be mechanically vibrated by means of a vibration motor.

A third aspect of the invention provides a method of therapeutic treatment comprising applying a pulsed and variable electro-magnetic field having a peak amplitude in the region of 1 G-100 G, advantageously at a frequency of between 1 kHz and 25 kHz, preferably approximately 20 kHz with a frequency modulation component preferably in the range from 1 Hz to 15 KHz.

In accordance with one aspect of the invention, there is provided a portable housing that generates a therapeutic magnetic field by means of electric current applied to at least two magnetic inductors that are supported within the housing whereby the magnetic inductors can provide a focused application of magnetic fields at least substantially adjacent in the inductor arrangement and whereby the inductor arrangement does not come into contact with the surface of the skin. Applicants have found that by the use of low power inductors, suitably directed towards an area to be treated, magnetic field effects can be provided in a focused fashion, whereby low power devices can be effectively utilised to provide a truly mobile magnetic field effect therapy device.

Conveniently, four inductors are placed in a compact device which can be placed either adjacent an area of skin or within a strap or cuff arrangement whereby the therapy can be directed to an area where required. Hydrogel adhesives may be attached to a housing so that a device in accordance with the invention can be securely placed in direct contact with a patient.

Preferably, the magnetic inductors comprise miniature inductors arranged and are mounted such that, in operation, their beams overlap. The device can be provided with inductors which are pivotally arranged about a pivot point whereby to enable adjustment of the distance of the convergence of the overlapping fields, so as to provide a variable magnetic focal field and provide a variable penetration of a magnetic field when in operation.

The magnetic inductors can be operated on a cyclic basis. The magnetic inductors can be operated in a sequential fashion. Conveniently, the housing includes a power supply, control electronics and user controls. Conveniently, a portable therapeutic device in accordance with the invention can provide pulsed magnetic fields between 0.5 KHz and 25 KHz. Preferably, the device operates in a frequency sweep from 20 KHz-1 KHz in a decaying sweep.

The present invention, by the provision of focused inductors, provides a truly portable pulsed magnetic field therapy device which is convenient and simple to use.

In a further form of the invention there is proposed a method of therapeutic treatment including the steps of: locating one or more applicator coils adjacent an area to be treated; applying electrical signals having frequency, amplitude and pulse characteristics to said applicator coils so as to produce multi-rhythmic, pulsed, alternating magnetic fields in the vicinity of the coils; and adjusting the frequency, amplitude and pulse characteristics so as to produce a pulsating magnetic field having therapeutic effect in the said area to be treated. Additionally, the unit may be vibrated by a vibration motor.

Further advantageous aspects of the invention will become apparent to those ordinarily skilled in the art upon review of the following description of a specific embodiment and with reference to the accompanying drawings.

### BRIEF DESCRIPTION OF THE DRAWINGS

To further assist in understanding the invention reference will be made to the following drawings in which:

FIG. 1 shows a first known prior art PEMF apparatus;

FIG. 2 shows a second known prior art PEMF apparatus;

FIG. 3 shown a first embodiment of the invention;

FIG. 4 shows a perspective view of an underside of the first embodiment;

FIG. 5 shows the first embodiment in plan view;

FIGS. 6a and 6b shows a side view of the device and a section thereof;

FIGS. 7a, 7b & 7c respectively show perspective, plan and sectional views of an exemplary inductor element;

FIG. 8 shows an exemplary use of a device in accordance with the invention within a holster upon limbs of a patient; and

FIG. 9 shows an exemplary use of a device in accordance with the invention where the device is adhesively secured to a patient.

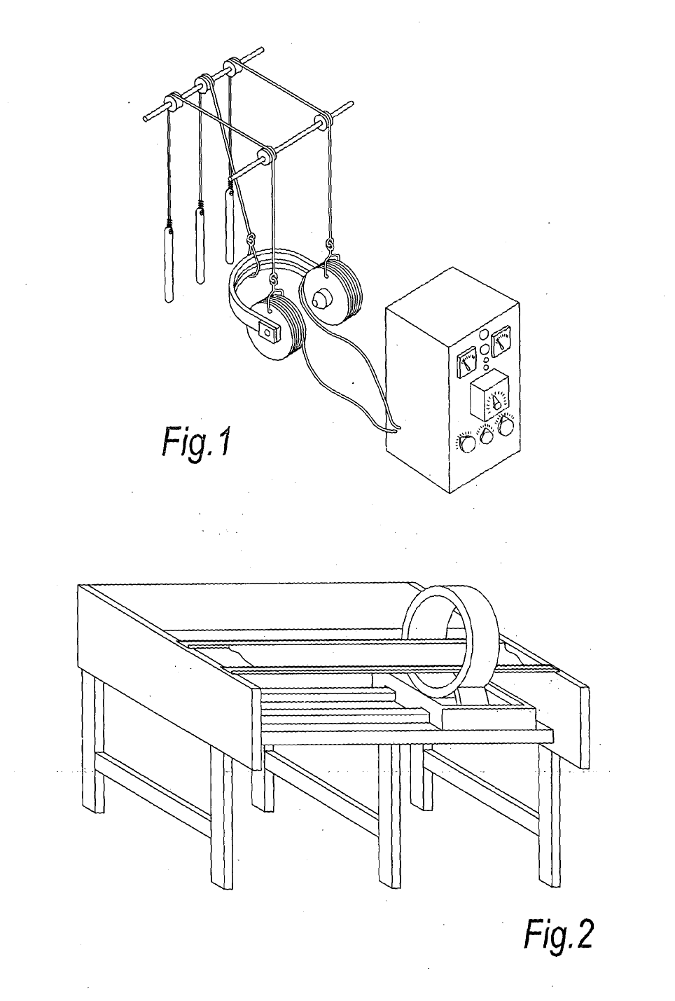

### DETAILED DESCRIPTION OF THE DRAWINGS

There will now be described, by way of example only, the best mode contemplated by the inventor for carrying out the present invention. In the following description, numerous specific details are set out in order to provide a complete understanding to the present invention. It will be apparent to those skilled in the art, that the present invention may be put into practice with variations of the specific.

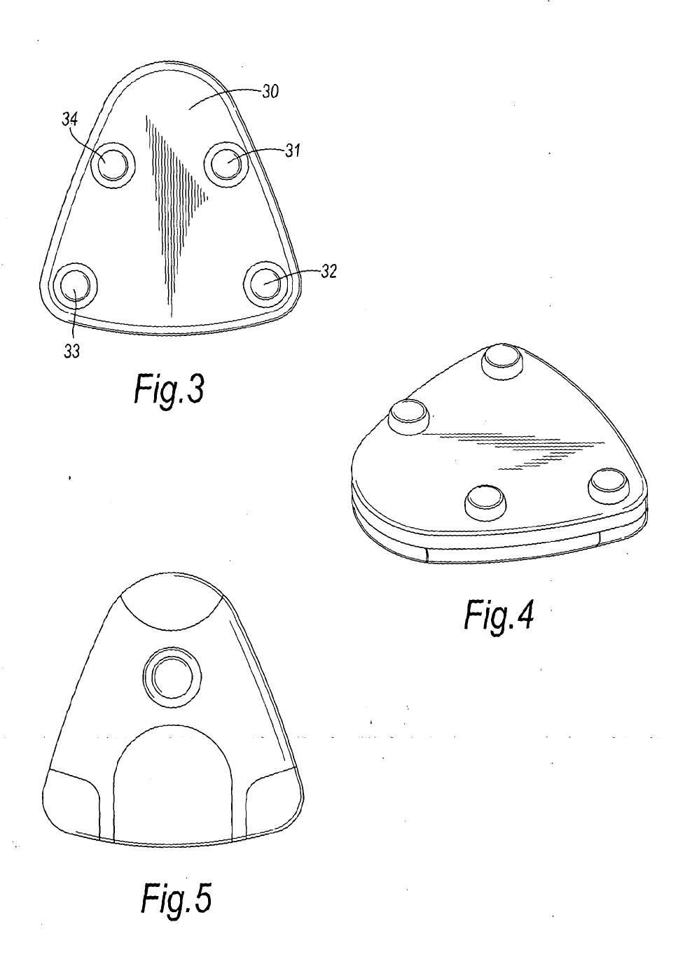

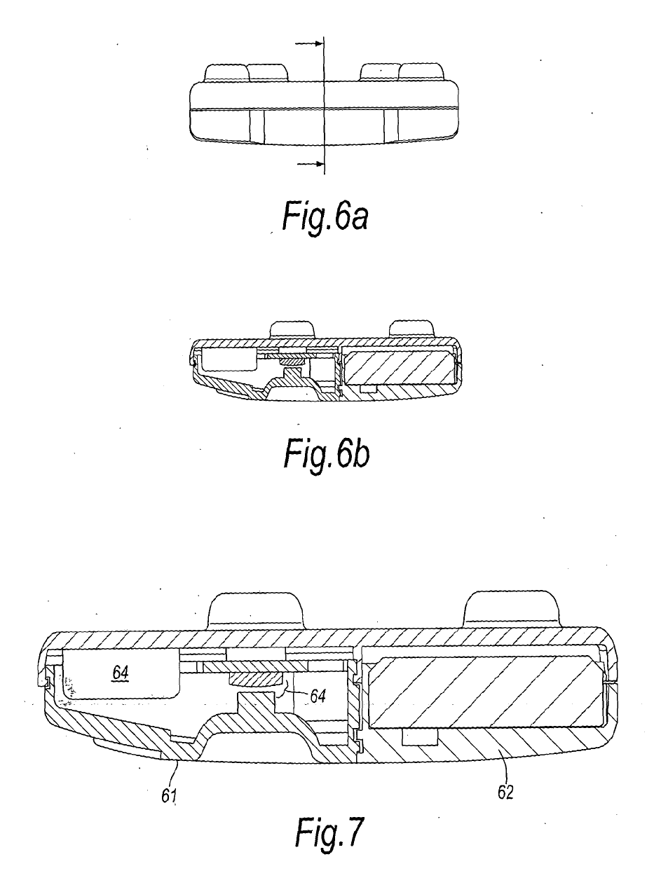

Referring now to FIG. 3, there is shown a first embodiment of the invention. The device comprises a generally triangular body 30, with one of the corners of the triangle being more acute than the other two. This has been proven to provide more adaptable device configuration, whereby the device can be placed snugly between limbs and/or folds of skin. Feet members 31-34 enable inductor elements (not shown) to be placed within recesses of an inside of the housing. These feet, in use, are placed against the body of a patient. FIG. 4 shows a perspective view of an underside—and to which the device would be placed against the body of a patient; FIG. 5 shows the device in plan view; FIG. 6a shows a view of the device from a side perpendicular to the acute corner and FIG. 6b shows a section on A-A of FIG. 6a. Reference numeral 61 indicates a soft polymeric plastics cover which has lip members associated with an outside edge member and is adapted to sealingly engage within inside lip 35 of housing 30. Conveniently the polymeric plastics member is anti-microbial. The polymeric cover has a depression which can be depressed to activate the device, by means of switch 63; a vibratory motor is indicated by reference 65. Low power control electronics are programmed to control the inductor elements powered by a 3 V lithium battery such as a CR2032, which has a diameter of 20 mm and a height of 3.2 mm, referenced 64. The total charge capacity for this class of electrical cell is typically 210 mAh and can supply an average current of 1 mA, with a peak current of 5 mA.

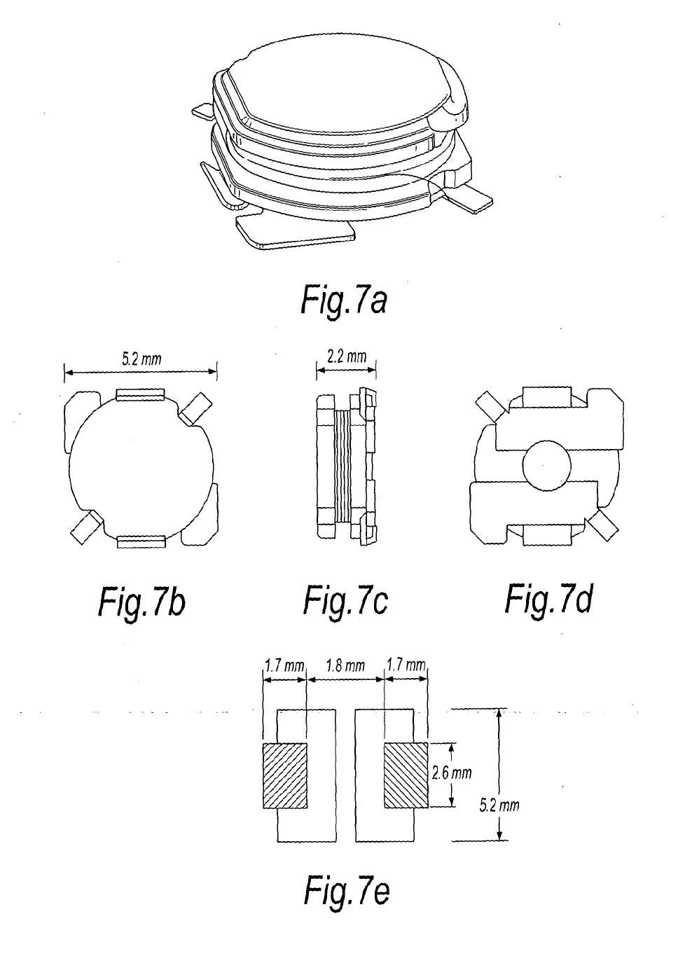

Turning now to FIG. 7a, an inductor element is shown. One inductor that has successfully been employed in tests is the 4.7 mH Toko #875FU inductor, which has a diameter of 5.2 mm, a depth of 2.2 mm. Whilst specified as drawing a current of 140 mA, at 100 KHz, in practice the average current is 1.5 mA and peak current is 5 mA. It will be appreciated that larger inductors can be employed: however, a greater current drain will also be realised, reducing the life expectancy of any electrical cell that is employed. The inductors are driven by a controller so that the operation is on a pulsed basis, The waveform is produced such that it changes from 1 KHz-20 KHz, on a decaying sweep. FIGS. 7b-7e show dimensions of such an inductor.

A typical mode of operation such as a 4 second duration comprising a series of 0.1 s on-0.2 s off bursts, each four second series being repeated in a range of times between 5 minutes and 30 minutes. It has been found to be surprising that such a low average excitation not only provides an effective pain relief regime but it is to be appreciated that such a low duty cycle significantly increases the life-time of an electrical cell; indeed since the running costs are low higher storage capacity non-rechargeable electrical cells may be employed as opposed to re-chargeable cells, which typically have a 30% greater capacity.

In a second embodiment, in addition to the magnetic inductors, a vibratory device 65 has been utilised to cause the device to oscillate to a degree once in operation. Typical vibration motors have an average current drain of 60-80 mA, with a 90-120 mA draw upon start-up. It has been found that not only can the oscillation provide a therapeutic benefit to the user of the device, the vibration can provide reassurance that the device is switched on the vibration pulses with magnetic pulse frequency. It is believed that the mild vibration not only defocuses the effective magnetic field or beam about a central core, but provides a mild physical stimulation which can assist in movement/stimulation of body tissue and body fluids in the vicinity of the device. Furthermore, the vibration motor can be programmed to operate upon the device being switched on and upon the device being switched off, whereby a clear indication of functioning can readily be determined by the user, without looking at the device—which might be convenient, when the device is worn under garments or is worn upon the back, for example.

In accordance with one duty cycle, the following operation is realised: a swept magnetic field is provided, from 1 kHz to 20 kHz, each complete sweep taking around 100 ms. This sweep is repeated every second and after 30 minutes duration the device will switch off.—Conveniently, the vibration motor generates several bursts of vibration at is intervals, this repeats every 5 minutes while the device is active.—Each burst of vibration lasts approx. 100 ms. When powered by a 3V cell with 220 mAh capacity—a proprietary “heavy duty” cell with low internal resistance, the electrical cell will last for more than 40 hours of treatment and more than twelve months in standby.

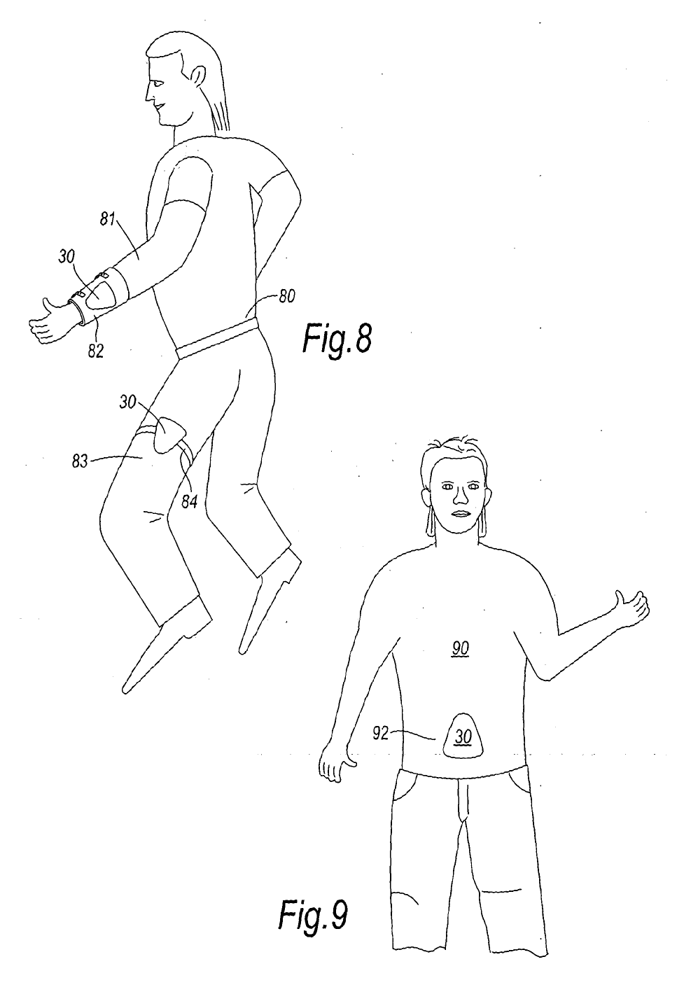

FIG. 8 shows how a device in accordance with the invention can be attached to a patient 80. Housing 30 of the invention is strapped to limbs of the patient—to the arms 81 by means of holster 82 and to the legs 83 by means of holster 84. The holster will be provided with straps that can elastically engage about a limb and be provided with a non-slip surface, whereby it remains in an area of placement, despite movement of the patient. FIG. 9 shows how hydrogel adhesive attached to the device 30 is placed upon the abdomen 92 of another patient 90. This method of application is particularly suitable for conditions such as period pain.

In a suitable electrical/electronic system, or device, suitable for generating the PEMF, a DC power source will be employed, using standard button cells or re-chargeable button-cells. A convenient power source would provide a DC voltage of, say, 2.8V and a current delivery of, say, 300 mA. A waveform generator, typically configured as a micro-processor operating under software control, or other suitably programmed processing device. The waveform generator function is to generate not only all the control functionality but also the specific detailed and unique wave shape(s) containing all the required frequency and amplitude components that are considered to be advantageous. The microprocessor (e.g. part no PIC 12F629) could suitably be programmed to accept/perform all power on/off functions and battery management for switching the device on and off during treatment.

A signal amplifier circuit can be used to amplify the waveform generated by the waveform generator to a preferred amplitude of 10 μT. This amplifier may for example comprise two power switches and a voltage multiplier chip U2 SP4561 voltage doubler may be used to boost the supply voltage to drive a larger current through a main inductor. Capacitor diode pairs can be used to generate this voltage. A high powered Field Effect transistor F.E.T such as an IRF530 N-channel power MOS transistor can be used to switch and amplify the generated waveform to a main inductor, being a reactive transformer having an inductance value of, for example, 4.7 MilliHenries. This can, for example, be a reactive component off the shelf or a planar etched inductance on the PCB. Diodes are conveniently included in the circuit as back EMF protectors to protect the circuit from reverse voltages generated by the inductor.

A signal is also fed to a vibration Motor including motor component. Conveniently, the circuit will be provided with diode-capacitor circuits to condition the supply voltage for M1 which serves the purpose of verifying the PEMF is present and provides user feedback that the system is operating.

The wave shapes generated have three important variables unlike conventional waveforms: Firstly, the amplitude is variable, e.g. programmable by the pulse generator; secondly, the carrier frequency is variable, e.g. programmable via the pulse generator; thirdly, the frequency of operation can be adjusted, preferably, between 1 Hz to 15 KHz, this is achieved by changing the variation of each cycle by a set value in the above range or other range, by, for example, a software algorithm. The advantage is that multiple PEMF frequencies can be delivered as an output of the system, i.e. a carrier frequency of, typically, 20 KHz with a modulated lower frequency of, by way of example, 5 Hz is delivered at the same time. It is also possible to extend this technique and change each pulse to different modulation frequencies so that a multiple low frequency PEMF can be delivered during a treatment. The preferred frequency modulation requires the oscillator frequency to deviate both above and below the carrier frequency. During the process of frequency modulation, the peaks of each successive cycle in the modulated waveform occur at times other than they would if the carrier were unmodulated. This is actually an incidental phase shift that takes place along with the frequency shift. The cycle varies in the modulated wave according to the added lower frequency wave variation. Since frequency is a function of time period per cycle, it may be seen that such a phase shift in the carrier will cause its frequency to change. The frequency change or shift is in phase with the added waveform. The amount of frequency change has nothing to do with the resultant modulated wave.

In some embodiments, the device may be arranged to operate in three or more modes of operation. By way of example, preferred electromagnetic waveforms generated in first, second a third modes of operation which may be referred respectively as the Ultra Low Frequency range, Very Low Frequency range and Low Frequency range modes.

A preferred output waveform for use with any of the aforementioned aspects of the invention can comprise a digitally modulated waveform. The centre carrier frequency could be, for example, approximately 21 KHz (although other frequencies are possible, especially those in the carrier frequency ranges given hereinbefore). The preferred amplitude is approximately 25 V decaying in digital steps to zero (although other amplitudes and/or decay rates are possible, especially those in the ranges given hereinbefore). The preferred pulse length is approximately 25 milliseconds, with a preferred delay of, say, approximately 50 ms between pulses for battery management. The waveform can be frequency modulated with one or more varying low frequency components sweeping from, for example,

The inventors have found that this specific sequence of ascending pulse frequencies provides even greater benefits than available from single frequency treatments, particularly in a reduction in the time needed to obtain relief and/or healing. The use of a microprocessor based control means allows for configuration of the device for a number of different applications. A timer buzzer 5 can provide an audible indication that a period of treatment has ended.

The device in a further embodiment can be provided with a liquid crystal display whereby a treatment regime can be indicated, and other useful information such as treatment time remaining and the frequency of treatment. Other indications, such as a program details can be shown if desired.

As mentioned above the waveform can be maintained at a lower average power by the non-simultaneous use of the inductors. The inventors have found through experimentation that the method and apparatus herein disclosed are useful for relief of pain, arthritis, rheumatism and back ache, for alleviating circulatory and respiratory problems, for reducing inflammation and for treating tendonitis and sports injuries. At frequencies between 12 KHz and 18 KHz the major blood vessels and capillaries are dilated, allowing increased blood flow which is necessary for the secondary stages of healing.

As is known, pulsating magnetic fields can alleviate pain sensations almost immediately. This is believed to arise from an increase in the oxygen partial pressure in the terminal tissue and the increase in the local perfusion and velocity of the capillary blood flow alleviating the accumulation of metabolites due to small vascularization and blood flow (transmitted by the sympathetic nervous system).

The inductors in accordance with the present invention can each produce magnetic field powers of 200 Gauss or more in small volume fields. By the superposition of at least two, conveniently four inductors, the present invention provides far greater magnetic field strengths than would otherwise be possible and, moreover, can enable such field strengths to be provided utilising small electrical cells, commonly referred to as button-cells. It will be appreciated that the frequency of the magnetic pulses, the strength of the fields can all be provided by selection of inductors—they need not all operate at the same time; they may operate, for example on an alternate basis or at alternate frequencies. In terms of cost of parts and manufacturing, different versions of the device can conveniently be fabricated employing a common housing shape, whereby cost per unit part can be reduced. For example a basic model may be provided with a fixed operating cycle that is known for a particular treatment; other models may have different programs, determined by the selection of a choice of push-buttons or through the selection of a treatment by timed entry of a single input button, the selection being confirmed, for example by the indication of one or more LED lights

This, together with the realisation that effective treatment can be provided without the powers that have negative biological effects. PEMF therapy is an absolutely heat-less therapy method, not a heat producing method, not a heat producing therapy. Therefore, all implants (except heart pacemakers) can be treated.

PEMF therapy can be employed to accelerate the healing of bones where pins and bone plates are employed; No damaging heat will be produced in the implants. Furthermore, all implants are antimagnetic. The treatment of fractures can also be applied with a plaster cast because, as mentioned before, magnetic fields permeate all materials. The therapeutic effect of treatment can last for approximately six to eight hours; by the use of truly mobile, low power PEMF device treatment periods can be considerably reduce since treatment is applied continuously.

PEMF has proven to be very effective including for osteoporosis treatment. Due to improved blood supply oxygen levels increase, activating and regenerating cells, resulting in improved calcium transport and better absorption of calcium in the bones and simultaneously decreases pain dramatically. In addition increase of bone density, results in improved fracture healing and less risk of braking bones.

Pulsating electromagnetic fields influence cell behaviour by inducing electrical changes around and within the cell. Improved blood supply increases the oxygen pressure, activating and regenerating cells. Increased calcium transport stimulates the repair and growth of cartilage while at the same time decreasing pain dramatically.

It has been found that the present invention can provide benefits in a number of conditions, for example: vasodilatation; the opening of blood vessels to increase blood pressure to the point of pain, allowing for more of the bodies chemicals to reach the point of pain and speeds up cell regeneration; and angiogenesis; This occurs in the formation of new blood vessels around the point of pain, again to increase blood pressure and increase the chemical flow; these effects are believed to assist in the speed up cell regeneration.

The frequency of the waveforms in the range of 1 KHz-20 KHz has been found to encourage vasodilatation and angiogenesis. As a result, it has been found that it is possible to influence the body's cell structure with small amplitude waveforms of 1 KHz/10 KHz to larger outputs of 11 KHz/20 KHz depending upon the bodies need.

The device has been designed especially to be worn using either, clothing, strapping or hydrogel double sided patches. The effect of the present invention is that the waveform empowers the body's cellular structure around the area or region of pain (inflammation) causing the cells to cooperate with each other and release the body's own chemicals such as the groups known as endorphins, enkephalins and others, which act to help respond to and reduce indications such as inflammation which causes pain and with absolutely no side effects. The present invention can also provide benefit as an acupuncture point stimulator; the magnetic fields can be utilised to provide the effects of needles, without the problems associated with needles themselves.

1. A portable therapeutic device comprising a housing, at least two magnetic inductors and control electronics, wherein the housing supports the magnetic inductors so that in operation they are spaced from an area to be treated, the magnetic inductors being arranged so that they can provide a focused application of magnetic fields at least substantially adjacent in a inductor arrangement and whereby the inductor arrangement does not come into contact with the surface of the skin.

2. A portable therapeutic device according to claim 1, wherein the housing includes a power supply, control electronics and user controls.

3. A portable therapeutic device according to claim 1, wherein the magnetic inductors comprise miniature inductors arranged and mounted such that, in operation, their beams overlap.

4. A portable therapeutic device according to claim 1, wherein the power supply comprises rechargeable electrical cells.

5. A portable therapeutic device according to claim 1, wherein the magnetic inductors are operated on a cyclic basis.

6. A portable therapeutic device according to claim 1, wherein the magnetic inductors are arranged to provide sequential operation.

7. A portable therapeutic device according to claim 1, wherein the magnetic inductors are arranged to provide pulsed magnetic fields between 1 and 100 Gauss at frequencies between 0.5 KHz and 25 KHz.

8. A portable therapeutic device according to claim 1, wherein the magnetic inductors are arranged to provide pulsed magnetic fields between 1 KHz and 20 KHz on a decaying sweep.

9. A portable therapeutic device according to claim 1, wherein there is a plurality of inductors arranged about a circle and the inductors are directed so that, in operation, their fields overlap about an axis from the circle.

10. A portable therapeutic device according to claim 1, wherein there is a plurality of inductors arranged about a circle and the inductors are directed so that, in operation, their fields overlap about an axis from the circle, wherein the inductors are pivotally arranged about a pivot point arranged generally parallel with an adjacent circumference to the circle, whereby to enable adjustment of the distance of the convergence of the overlapping fields, to provide a variable magnetic focal field about said axis.

11. A portable therapeutic device according to claim 1, wherein there is provided adhesive means whereby the housing may be adhesively secured to an area of skin or other epidermis whereby to enable the housing to be securely retained adjacent a users body.

12. A portable therapeutic device according to claim 1, wherein there is provided one of a buckle, hoop or clip whereby to enable the housing to be securely retained adjacent a users body.

13. A portable therapeutic device according to claim 1, wherein there is provided one of a holster or retaining strap whereby to enable the housing to be securely retained adjacent a users body.

14. A portable therapeutic device according to claim 1, wherein the housing includes a power supply, control electronics and user controls, wherein there is provided a power jack, whereby an additional source of power may be utilised, for extended usage of the device.

15. A portable therapeutic device according to claim 1 wherein the dimensions of the body of the device are no more than 50×50×6 mm.
